(19) United States
(12) Patent Application Publication
(10) Pub. No.: US 2010/0273726 A1
(43) Pub. Date: Oct. 28, 2010

(54) METHICILLIN-RESISTANT STAPHYLOCOCCUS AUREUS ACTIVE METABOLITES ISOLATED FROM LAURUS NOBILIS AND COMBINATIONS THEREOF

(75) Inventors: Mark T. Hamann, Oxford, MS (US); Mohamed A. Ibrahim, Oxford, MS (US)

Correspondence Address: Baker Donelson Bearman, Caldwell & Berkowitz, PC, 920 Massachusetts Ave, NW, Suite 900, Washington, DC, 20001, US

(73) Assignee: TRITON BIOPHARMA, Oxford, MS (US)

(21) Appl. No.: 12/767,944

(22) Filed: Apr. 27, 2010

Related U.S. Application Data
(60) Provisional application No. 61/173,026, filed on Apr. 27, 2009.

(51) Int. Cl. A61K 31/7048; C07H 17/04; A61P 31/04

(52) U.S. Cl. 514/27; 536/16.8

(57) ABSTRACT

This present provides kaempferol-3-(2-E,3-Z-di-p-coumaroyl)-rhamnoside, kaempferol-3-(2-Z-3-E-di-p-coumaroyl)-rhamnoside, and kaempferol-3-(2,3-di-Z-p-coumaroyl)-rhamnoside compounds useful as a new class of anti-bacterial agents. These compounds are extracted from Platanus occidentalis or Laurus nobilis. These compounds were found to exhibit anti-bacterial activity against methicillin resistant Staphylococcus aureus (MRSA).

### FIELD OF THE INVENTION

The present invention relates to a new class of compounds extracted from various plant species such as Laurus nobilis and Platanus occidentalis as well as combinations of these extracts. These compounds were found to exhibit anti-bacterial activity against methicillin resistant Staphylococcus aureus (MRSA).

### BACKGROUND

Methicillin-resistant Staphylococcus aureus (MRSA) is a serious pathogen that causes patient mortality. Over 50% of infections around the world are caused by MRSA. MRSA has emerged as a community-associated pathogen (CA-MRSA), usually resulting in skin infections with abscess formation and cellulites in individuals who have not been hospitalized within the last year. Moreover, hospital-acquired infections of MRSA (HA-MRSA), relative to CA-MRSA, have been shown to be resistant to multiple antibiotics.

Two mechanisms of resistance of MRSA to the β-lactam antibiotics are inactivation by β-lactamase and the production of a penicillin binding protein PBP2a with decreased affinity for the antibiotics. The resistance PBP2a genotype of MRSA strains is encoded mecA gene which is transported on a mobile genetic element known as a staphylococcal cassette chromosome (SCC). The differences in susceptibility of HA-MRSA and CA-MRSA can be attributed to different SCCmec types (types IV and V for CA-MRSA and types I, II and III for HA-MRSA). These genes are associated with resistance to multiple drug classes, in addition to β-lactam antibiotics. CA-MRSA isolates are primarily resistant to β-lactam antibiotics (penicillins, cephalosporins, carbapenems) and macrolides where initiation of antibiotic therapy may not be necessary in all patients with skin and soft tissue infections caused by CA-MRSA.

Vancomycin has been the drug of choice despite a success rate of 35-57% and side effects including nosocomial pneumonia, skin and soft tissue infections, in addition to low bone penetration limit vancomycin's utility. Moreover, the increased use of vancomycin, especially in chronic patients has resulted in the emergence of MRSA with reduced susceptibility to glycopeptides.

The most effective anti-MRSA drug used today is daptomycin, a cyclic lipopeptide derived from the fermentation of Streptomyces roseosporus. Daptomycin is most useful for use in short duration and to treat persistent MRSA unaffected by other drug treatments such as vancomycin. Its mechanism of action involves binding to the bacterial cell membrane, causing depolarization of the membrane potential leading to inhibition of protein, DNA and RNA synthesis. Unfortunately, the FDA has reported some side effects for daptomycin, including an increase in blood creatine phosphokinase, rhabdomyolysis, skin exfoliation and skin ulcers.

Efforts have been made to decrease infection and colonization of MRSA in hospitals and other environments including the use of products that sterilize surfaces, including hand washes. Products containing 4% chlorhexidine gluconate (CHG) and 1% triclosan reduce the total bacterial count. The 4% CHG is more effective at reducing the total count than 1% triclosan; however, 1% triclosan has the ability to remove MRSA while 4% CHG cannot. Povidone-iodine used as an intranasal cream by the physicians and nurses working in the neonatal intensive care unit shows a 10% reduction in the isolation of MRSA pathogens. Because of the increasing presence of MRSA-related infections and the lack of acceptable antibiotic therapy, new antibacterial compounds are needed.

Laurus nobilis is known as Bay Laurel or True Laurel, Sweet Bay, Laurel Tree, Grecian Laurel, Laurel, or Bay Tree. It is an aromatic evergreen tree or large shrub reaching 10-18 meters tall, and is native to the Mediterranean region.

The leaves are 6-12 cm long and 2-4 cm broad, with a characteristic finely serrated and wrinkled margin. It is dioecious, with male and female flowers on separate plants; each flower is pale yellow-green, about 1 cm diameter, borne in pairs together beside a leaf. The fruit is a small black berry about 1 cm long, containing a single seed.

Bay Laurel is the source of the bay leaves which are used for their flavor in cooking. The aromatic laurel leaves are used in many countries of central and southern Europe to flavor stews, soups, meat dishes and even fish. It is also widely cultivated as an ornamental plant in regions with Mediterranean or oceanic climates, and as an indoor plant in colder regions.

Some evidence from the medical literature supports Bay Laurel having these uses: antioxidative (Fitoterapia. 2003 September; 74(6):613-6); analgesic and anti-inflammatory (Phytother Res. 2003 August; 17(7):733-6); and an anticonvulsant (antiepileptic) (Phytomedicine. 2002 April; 9(3):212-6).

In the fruit there are essential oils and fatty oils present. The fruit is pressed and water extracted to obtain these products. The fruit contains up to 30% fatty oils and about 1% essential oils (terpenes, sesquiterpenes, alcohols and ketones). The leaves contain about 1.3% essential oils (Ol. Lauri folii), consisting of 45% eucalyptol, 12% terpenes, 3-4% sesquiterpenes, 3% methyleugenol and other α- and β-pinenes, phellandrene, linalool, geraniol and terpineol.

Platanus occidentalis, the American Sycamore (fam. Platanaceae) has been known for its high safety profile in the treatment of a wide variety of conditions in traditional folk medicine. Species of Platanaceae have been used frequently for their antimicrobial and antiseptic properties. Native Americans used the American sycamore for cold and cough remedies, as well as dietary, dermatological, gynecological, respiratory, and gastrointestinal aids. The bark was used with honey locust to relieve hoarseness and sore throats as well as to treat skin eruptions, scabs and eczema, lung troubles, hemorrhages and tuberculosis. The mixture of the bark with the stem and twigs was used to treat knife and axe wounds. In addition, the bark has been used to treat colds, for purifying blood, for weight gain and as an analgesic. Finally, sycamore was also taken as cathartic, emetic and anti-diarrheal drug to treat dysentery.

Platanus occidentalis L. is a massive perennial tree up to 50 m in height, up to 4 m in diameter and usually found near lakes and streams. Several chemical investigations have shown the presence of triterpene secondary metabolites. This species is also reported to produce betulinic aldehyde, betulinic acid, platanic acid, β-sitosterol, and tiliroside as well as kaempferol 3-O-rhamnosides. Moreover, some polar and non-polar glycosides have been reported from members of the Platanaceae family.

Because of the increasing presence of MRSA-related infections and the lack of acceptable antibiotic therapy, new antibacterial compounds are needed.

### SUMMARY

The present invention provides compounds extracted from various plant species, such as, but not limited to Laurus nobilis, Platanus occidentali, Archeria traversii, Dracophyllum acerosum, Dracophyllum latifolum, Dracophyllum lessonianum, Dracophyllum longifolium varieties, Dracophyllum sinclairii, Dracophyllum uniflorum, Epacris alpina, Epacris pauciflora, Cyathodes empetrifolia, Leucopogon fasciculatus, Leucopogon fraserii, Leucopogon suavolens, and Pentachondra pumila and mixtures of these compounds from one plant species as well as mixtures of these compounds extracted from more than one plant species. In one embodiment, the compounds are extracted from Platanus occidentalis. In another embodiment, these compounds are extracted from Platanus occidentali. These compounds were found to exhibit anti-bacterial activity against methicillin resistant Staphylococcus aureus (MRSA).

The compounds of the invention have the general formula I shown below:

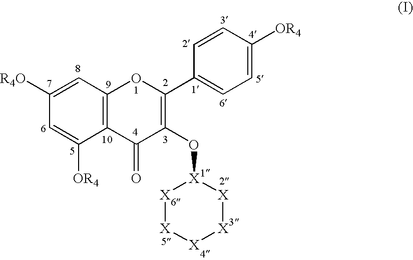

wherein X_{1″}, X_{2″}, X_{3″}, X_{4″}, X_{5″}, X_{6″} are independently C, COR₁, COR₂, COR₃, CY, N, S, or O;
wherein Y is CH₃;
wherein at least one of X_{1″}, X_{2″}, X_{3″}, X_{4″}, X_{5″}, X_{6″} is N, S or O;
wherein R₁, R₂ and R₃ are independently, hydrogen, halogen, hydroxyl, C₆ alkyl, C₁-C₆ alkoxy, C₁-C₆ acyloxy, mono, di or tri-C₁-C₆ alkyl, mono, di or tri-C₁-C₆ N-alkyl, C₁-C₆ alkylthiol, C₁-C₆ acylamino, thiol, nitro, amino, C₁-C₆ alkylsulfonyl, aminosulfonyl, hydroxy sulfonyl (—SO₃H), E-p-coumaroyl, Z-p-coumaroyl;

wherein E-p-coumaroyl has the formula:

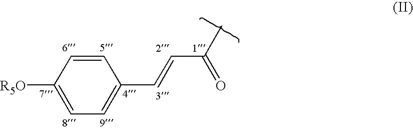

and wherein Z-p-coumaroyl has the formula:

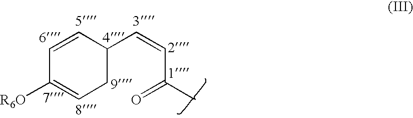

wherein R₄, R₅ and R₆ are independently hydrogen, halogen, hydroxyl, C₆ alkyl, C₁-C₆ alkoxy, C₁-C₆ acyloxy, mono-C₁-C₆ alkyl, di-C₁-C₆N-alkyl, C₁-C₆ alkylthiol, C₁-C₆ acylamino, thiol, nitro, amino, C₁-C₆ alkylsulfonyl, aminosulfonyl, hydroxy sulfonyl (—SO₃H).

The present invention further provides compounds having formula I wherein: R₁, R₂ and R₃ are independently E-p-coumaroyl, Z-p-coumaroyl, hydrogen, CH₃, C₂H₅, C₃H₇, C₄H₉, C₅H₁₁, C₆H₁₃, C₆H₁₀, C₂H₃O₂, C₃H₅O₂, trifluoromethanesulfonate (CF₃—SO₃—) or benzoyl (C₆H₅—C(═O—); and wherein R₄, R₅ and R₆ are independently hydrogen, hydrogen, CH₃, C₂H₅, C₃H₇, C₄H₉, C₅H₁₁, C₆H₁₃, C₆H₁₀, C₂H₃O₂, C₃H₅O₂, trifluoromethanesulfonate (CF₃—SO₃—) or benzoyl (C₆H₅—C(═O—).

In certain embodiments, the compounds of the present invention have the formula I wherein R₃ and R₄ are hydrogen and wherein R₁ represents E-p-coumaroyl and R₂ represents Z-p-coumaroyl; or R₁ represents Z-p-coumaroyl and R₂ represents E-p-coumaroyl; or R₁ and R₂ represent Z-p-coumaroyl. One compound of the present invention has formula I wherein R₃ and R₄ are hydrogen and R₁ represents E-p-coumaroyl and R₂ represents Z-p-coumaroyl. Another compound of the invention has formula I wherein R₃ and R₄ are hydrogen and wherein R₁ represents Z-p-coumaroyl and R₂ represents E-p-coumaroyl. Another compound of the invention has formula I wherein R₃ and R₄ are hydrogen and wherein R₁ and R₂ represent Z-p-coumaroyl.

In another embodiment of the invention compounds extracted from Laurel nobilis have the formula V where

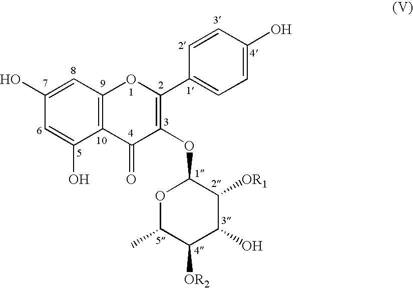

wherein

R₁ represents E-p-coumaroyl and R₂ represents Z-p-coumaroyl; or

R₁ and R₂ represent Z-p-coumaroyl.

The present invention provides a composition comprising the compounds of formula V wherein
R₁ and R₂ represents E-p-coumaroyl;
R₁ represents E-p-coumaroyl and R₂ represents Z-p-coumaroyl;
R₁ represents Z-p-coumaroyl and R₂ represents E-p-coumaroyl; and
R₁ and R₂ represent Z-p-coumaroyl.

The present invention provides a composition comprising at least one compound of Formula I and at least one compound of Formula V. In another embodiment, the present invention provides a composition comprising at least one compound of Formula IV and at least one compound of Formula V. Another embodiment of the present invention provides a composition comprising (or consisting essentially of) each of the following compounds:

a) compounds having the formula IV wherein
R₁ represents E-p-coumaroyl and R₂ represents Z-p-coumaroyl;
R₁ represents Z-p-coumaroyl and R₂ represents E-p-coumaroyl; and
R₁ and R₂ represent Z-p-coumaroyl; and

b) compounds having the formula V wherein
R₁ represents E-p-coumaroyl and R₂ represents Z-p-coumaroyl;
R₁ and R₂ represent Z-p-coumaroyl.

The present invention provides compounds of the formula of I, IV or V isolated from any plant species, or made synthetically. Exemplary plant species include, but are not limited to, Laurus nobilis, Platanus occidentali, Archeria traversii, Dracophyllum acerosum, Dracophyllum latifolum, Dracophyllum lessonianum, Dracophyllum longifolium varieties, Dracophyllum sinclairii, Dracophyllum uniflorum, Epacris alpina, Epacris pauciflora, Cyathodes empetrifolia, Leucopogon fasciculatus, Leucopogonfraserii, Leucopogon suavolens, and Pentachondra pumila. In certain embodiments, the compounds have the formula I, IV or V where:
R₁ and R₂ represents E-p-coumaroyl;
R₁ represents E-p-coumaroyl and R₂ represents Z-p-coumaroyl;
R₁ represents Z-p-coumaroyl and R₂ represents E-p-coumaroyl; and
R₁ and R₂ represent Z-p-coumaroyl.

The present invention also provides a method of treating bacterial infection comprising administering an effective amount of a compound of the present invention or a composition of the present invention.

In certain embodiments, a composition comprising a compound of formula I wherein R₃ and R₄ are hydrogen and wherein R₁=R₂=E-p-coumaroyl is administered to treat a bacterial infection.

The bacteria infection may be caused by Staphylococcus aureus. The Staphylococcus aureus may be methicillin-resistant, vancomycin-resistant or any other drug resistant strain.

The present invention further provides a pharmaceutical composition comprising at least one compound or mixture of compounds of the invention in a pharmaceutically acceptable carrier thereof.

Compounds of the invention may also be used as α-glucosidase inhibitors, which may provide a method of treatment of type II diabetes.

The present invention further provides a method of extracting compounds of the invention from a sycamore tree and/or its parts (for example, from leaves, bark, twigs and small branches).

The present invention also provides a topical or surface disinfectant or antiseptic comprising at least one compound of the present invention alone or in combination with an additional herbal or disinfectant or antiseptic substance. The antiseptic may be used on an animal (i.e. mammal, bird, reptile, etc.) or surface.

### BRIEF DESCRIPTION OF FIGURES

FIG. 1 is a table showing in vitro antimicrobial activity for isolated glycosides and compounds of the present invention compared with known antibiotics.

FIG. 2A is a chart that shows the effect of compound 1 on MRSA thigh infection in a murine model.

FIG. 2B is a chart that shows the percent reduction in infection after treatment with compound 1.

FIG. 3 is a chart that provides results from in vitro cytotoxicity testing.

FIG. 4 provides exemplary compounds of the present invention.

FIG. 5 provides exemplary compounds of the present invention extracted from Platanus occidentalis.

FIG. 6 provides exemplary compounds of the present invention extracted from Laurus nobilis.

### DETAILED DESCRIPTION COMPOUNDS

The present invention provides compounds having the general formula I shown below:

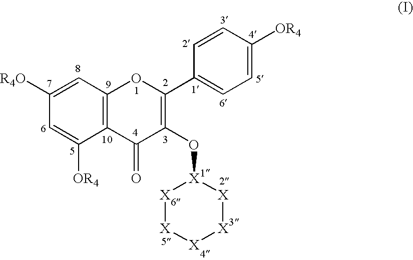

wherein X_{1″}, X_{2″}, X_{3″}, X_{4″}, X_{5″}, X_{6″} are independently C, COR₁COR₂, COR₃, CY, N, S, or O;
wherein Y is CH₃;
wherein at least one of X_{1″}, X_{2″}, X_{3″}, X_{4″}, X_{5″}, X_{6″} is N, S or O;
wherein R₁, R₂ and R₃ are independently, hydrogen, halogen, hydroxyl, C₆ alkyl, C₁-C₆ alkoxy, C₁-C₆ acyloxy, mono, di or tri-C₁-C₆ alkyl, mono, di or tri-C₁-C₆ N-alkyl, C₁-C₆ alkylthiol, C₁-C₆ acylamino, thiol, nitro, amino, C₁-C₆ alkylsulfonyl, aminosulfonyl, hydroxy sulfonyl (—SO₃H), E-p-coumaroyl, Z-p-coumaroyl;

wherein E-p-coumaroyl has the formula:

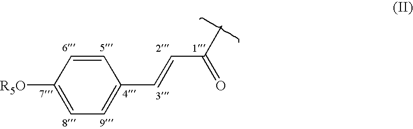

and wherein Z-p-coumaroyl has the formula:

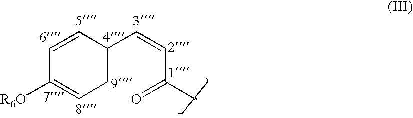

wherein R₄, R₅ and R₆ are independently hydrogen, halogen, hydroxyl, C₆ alkyl, C₁-C₆ alkoxy, C₁-C₆ acyloxy, mono-C₁-C₆ alkyl, di-C₁-C₆ N-alkyl, C₁-C₆ alkylthiol, C₁-C₆ acylamino, thiol, nitro, amino, C₁-C₆ alkylsulfonyl, aminosulfonyl, hydroxy sulfonyl (—SO₃H).

In certain embodiments, R₁, R₂ and R₃ are independently E-p-coumaroyl, Z-p-coumaroyl, hydrogen, CH₃, C₂H₅, C₃H₇, C₄H₉, C₅H₁₁, C₆H₁₃, C₆H₁₀, C₂H₃O₂, C₃H₅O₂, trifluoromethanesulfonate (CF₃—SO₃—) or benzoyl (C₆H₅—C(═O—); and wherein R₄, R₅ and R₆ are independently hydrogen, hydrogen, CH₃, C₂H₅, C₃H₇, C₄H₉, C₅H₁₁, C₆H₁₃, C₆H₁₀, C₂H₃O₂, C₃H₅O₂, trifluoromethanesulfonate (CF₃—SO₃—) or benzoyl (C₆H₅—C(═O—).

In other embodiments, compounds of the present invention have the formula I or IV where

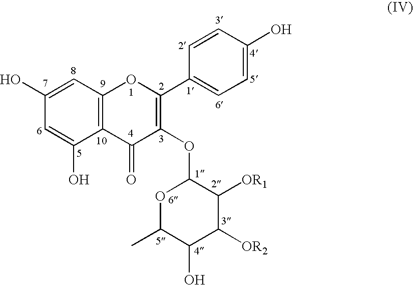

wherein R₃ and R₄ are hydrogen and wherein R₁ represents E-p-coumaroyl and R₂ represents Z-p-coumaroyl; or R₁ represents Z-p-coumaroyl and R₂ represents E-p-coumaroyl; or R₁ and R₂ represent Z-p-coumaroyl. Where R₁ and R₂ are E-p-coumaroyl, the present inventors refer to this compound herein as “compound 1.”

In a preferred embodiment the compound is kaempferol-3-(2-E,3-Z-di-p-coumaroyl)-rhamnoside (referred to herein as compound 2), kaempferol-3-(2-Z-3-E-di-p-coumaroyl)-rhamnoside (referred to herein as compound 3), or kaempferol-3-(2,3-di-Z-p-coumaroyl)-rhamnoside (referred to herein as compound 4).

It is understood that the OR₁, OR₂ and/or OR₃ groups off of any of X_{1″}, X_{2″}, X_{3″}, X_{4″}, X_{5″}, X_{6″} can be orientated in either the R or S orientation. Any pyranose (6-membered ring having 5 carbons and one oxygen) can be used in the synthesis of these compounds.

The present invention also provides compounds of formula V

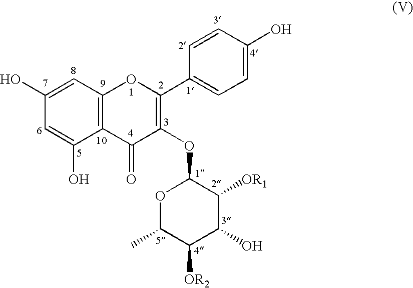

wherein

R₁ represents E-p-coumaroyl and R₂ represents Z-p-coumaroyl; or

R₁ and R₂ represent Z-p-coumaroyl.

Preferred compounds are of the formula I, IV or V where

R₁ represents Z-p-coumaroyl and R₂ represents E-p-coumaroyl;

R₁ represents E-p-coumaroyl and R₂ represents Z-p-coumaroyl; or

R₁ and R₂ represent Z-p-coumaroyl.

The present invention provides compounds of the formula of I, IV or V isolated from any plant species, or made synthetically. Exemplary plant species include, but are not limited to, Laurus nobilis, Platanus occidentali, Archeria traversii, Dracophyllum acerosum, Dracophyllum latifolum, Dracophyllum lessonianum, Dracophyllum longifolium varieties, Dracophyllum sinclairii, Dracophyllum uniflorum, Epacris alpina, Epacris pauciflora, Cyathodes empetrifolia, Leucopogon fasciculatus, Leucopogon fraserii, Leucopogon suavolens, and Pentachondra pumila. In certain embodiments, the compounds have the formula I, IV or V where:
R₁ and R₂ represents E-p-coumaroyl;
R₁ represents E-p-coumaroyl and R₂ represents Z-p-coumaroyl;
R₁ represents Z-p-coumaroyl and R₂ represents E-p-coumaroyl; and
R₁ and R₂ represent Z-p-coumaroyl.

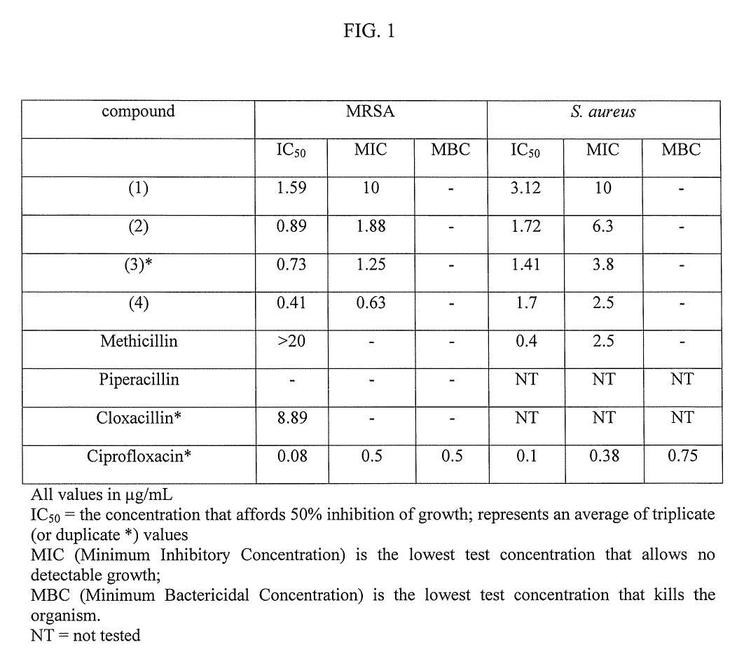

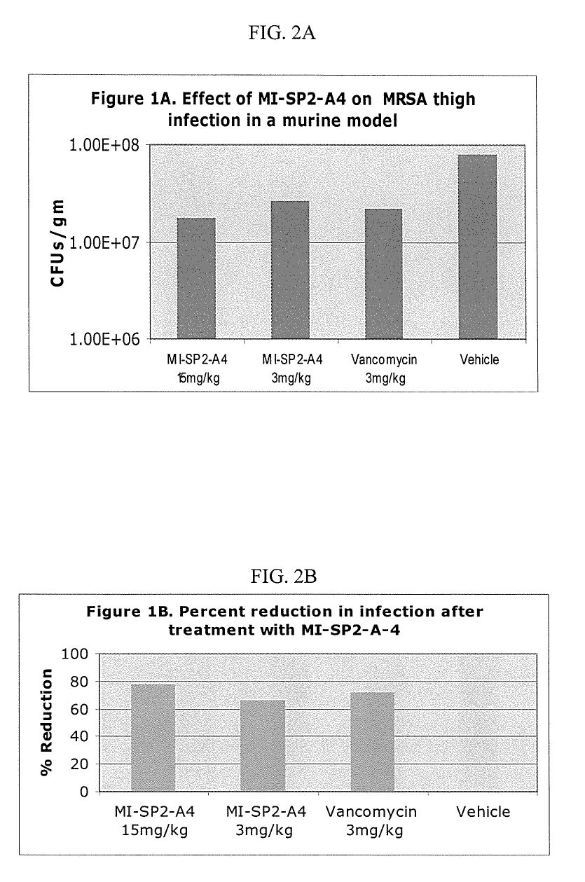

Ethanolic extracts of various parts of Platanus occidentalis were subjected to antimicrobial evaluation, amongst which leaves were found to be non-toxic, highly active and selective against MRSA. Four active compounds (1, 2, 3 and 4) were isolated with the molecular weight 725.2282 [M+H]⁺, molecular formula (C₃₉H₃₂O₁₄), and were shown by ¹H-NMR and ¹³C-NMR to be rhamnose glycosides with different configurations around the rhamnose or olefinic moiety. Results of in vitro evaluation of four compounds of the present invention is provided in FIG. 1. In vivo evaluation of one compound of the present invention is represented in FIGS. 2A and 2B.

Antimicrobial evaluation of the extracts of various parts of Platanus occidentalis showed that sycamore leaves contained antibacterial compounds, and were thus purified using bioassay-guided isolation against MRSA. Further fractionation and purification of the ethyl acetate:MeOH (75:25) fraction using HPLC resulted in the isolation of four active metabolites; the structure elucidation for all isolated compounds were performed using LCMS and NMR spectroscopy. Final structure confirmation was accomplished by comparing ¹H and ¹³C NMR data with that reported in the literature.

¹H NMR spectra of compounds 1, 2, 3 and 4 clearly displayed the characteristics signals of the kaempferol nucleus and the α-configuration of the rhamnoside sugar. High resolution TOF-ESI-MS of compounds 1, 2, 3 and 4 provided a molecular mass of [M+H]⁺ at m/z 725.2282 corresponding to the molecular formula (C₃₉H₃₂O₁₄). A search using SciFinder Scholar for comparison of ¹H NMR and ¹³C-NMR data with the reference proved that the major glycoside is known as kaempferol 3-O-α-L-(2″,3″-di-E-p-coumaroyl)rhamnoside (1) or commonly called platanoside in addition to three new anti-MRSA compounds: kaempferol 3-O-α-L-(2″-E-p-coumaroyl-3″-Z-p-coumaroyl)rhamnoside (2), kaempferol 3-O-α-L-(2″-Z-p-coumaroyl-3″-E-p-coumaroyl)rhamnoside (3), and kaempferol 3-O-α-L-(2″,3″-di-Z-p-coumaroyl)rhamnoside (4).

The UV, ¹HNMR, ¹³CNMR of compounds 2, 3 and 4 are highly similar to compound (1). Investigation of HMBC and COSY correlations indicated the positions of attachments of both p-coumaroyl units to L-rhamnose while ¹H NMR coupling constants and NOESY spectra revealed the configurations at C-2″′, C-3″′ and C-2″″, C-3″″ for the isolated metabolites 2-4. HMBC correlations between H-2″′ (5.81 Hz), C-1″′ (166.6 Hz) as well as H-3″ (5.27 Hz), C-1″′ (167.3 Hz) and the presence of the COSY correlation between H-2″ (5.81 Hz), H-3″ (5.27 Hz) confirmed the attachment of both p-coumaroyl units to position 2 and 3. The large coupling constant between H-2″′ and H-3″′ (15.6 Hz) in the known compound, 1 indicated the trans relationship at both p-coumaroyl units attached to both positions 2 and 3 at L-rhamnose. For glycoside 2, HMBC correlations between H-2″ (5.78 Hz), C-1″′ (166.4 Hz) as well as H-3″ (5.27 Hz), C-1″″ (166.1 Hz) and the presence of the COSY correlation between H-2″ (5.78 Hz), H-3″ (5.27 Hz) confirmed the attachment of both p-coumaroyl units to position 2 and 3. The J value between H-2″′ and H-3″′ (16.0 Hz) of the p-coumaroyl functionality at position 2 of the L-rhamnose indicated an E configuration while the J value between H-2″″ and H-3″″ (12.8 Hz) on the p-coumaroyl attached to position 3 of the L-rhamnose indicated a Z configuration, which was also confirmed by the NOE correlation between H-2″″ and H-3″″. The opposite arrangement was shown for glycoside 3, where HMBC correlations between H-2″ (5.78 Hz), C-1″″ (165.2 Hz) as well as H-3″ (5.27 Hz), C-1″′ (167.3 Hz) and the presence of the COSY correlation between H-2″ (5.78 Hz), H-3″ (5.27 Hz) confirmed the attachment of both p-coumaroyl units to position 2 and 3. The J value between H-2″′ and H-3″′ (16.0 Hz) of the p-coumaroyl functionality at position 3 of L-rhamnose supported the E relationship, while the J value between H-2″″ and H-3″″ (12.8 Hz) of the p-coumaroyl functionality at position 2 of L-rhamnose suggested a Z-relationship, which was also confirmed by the NOE correlation between H-2″″ and H-3″″. HMBC correlations in glycoside 4 between H-2″ (5.79 Hz), C-1″″ (166.1 Hz) as well as H-3″ (5.21 Hz), C-1″″ (166.2 Hz) and the presence of the COSY correlation between H-2″ (5.79 Hz), H-3″ (5.21 Hz) confirmed the attachment of both p-coumaroyl units to positions 2 and 3. The small coupling constants (12.8 Hz) for glycoside 4 between H-2″″ and H-3″″ established the Z configuration¹⁸ of both p-coumaroyl groups at positions 2 and 3 of L-rhamnose; this was also confirmed by the NOE correlations between H-2″″ and H-3″″. The absolute configuration of the sugar was showed to be L-rhamnose by GC comparison of its acetylated thiazolidine derivative with that of an L-rhamnose standard. See Ibrahim et al, Methicillin-resistant Staphylococcus aureus (MRSA)-active Metabolites from Platanus occidentalis (American Sycamore), herein incorporated by reference in its entirety.

The in vitro antimicrobial activity for the isolated glycosides is represented in FIG. 1. A modified version of CLSI (formerly NCCLS) method was used for the in vitro evaluation of test samples. Duplicate samples were transferred to 96-well microplates after diluting with 0.9% saline. The microbial cell suspensions were to give the desired target inocula after addition to the samples. Media and solvent controls were included in each assay. The IC₅₀ was calculated by plotting percent growth versus test concentration. Of the four compounds, compound (4) showed the most potent in vitro activity, indicating that the configuration of the double bond of the coumaroyl moiety is important for bioactivity. All compounds were inactive against other test organisms including the Gram negative bacteria E. coli and Pseudomonas aeruginosa, as well as against Mycobacterium intracelluare, in addition to the fungi Candida albicans, Candida glabrata, Candida krusei, Cryptococcus neoformans and Aspergillus fumigatus at the highest test concentration of 100 μg/mL. These compounds demonstrate a unique specificity for MRSA.

### Compositions

The present invention also provides compositions comprising a compound of the present invention. The term “comprising” as used herein in the detailed description is meant to convey that other embodiments of the invention may also either “consist essentially of” or “consist of.”

In certain embodiments, the composition comprises at least two compounds of the present invention. In other embodiments the composition may comprise at least 2 to 6 compounds of the present invention. In other embodiments, the composition comprises more than 2 compounds of the present invention. It may be beneficial to have more than one compound of the invention in the composition to prevent or delay the development of resistant strains. In certain embodiments, the composition comprises a mixture of compounds extracted from different sources, such as from, but not limited to Laurus nobilis and Platanus occidentalis Archeria traversii, Dracophyllum acerosum, Dracophyllum latifolum, Dracophyllum lessonianum, Dracophyllum longifolium varieties, Dracophyllum sinclairii, Dracophyllum uniflorum, Epacris alpina, Epacris pauciflora, Cyathodes empetrifolia, Leucopogon fasciculatus, Leucopogon fraserii, Leucopogon suavolens, and Pentachondra pumila. For example, the composition may comprise at least one compound of the formula IV and at least one compound of the formula V. In other embodiment, the composition may comprise compounds isolated from different plant sources, for example, compounds isolated from Laurus nobilis and Platanus occidentalis. In one embodiment, the composition comprises each of the following compounds:

a) compounds having the formula IV wherein
R₁ and R₂ represents E-p-coumaroyl;
R₁ represents E-p-coumaroyl and R₂ represents Z-p-coumaroyl;
R₁ represents Z-p-coumaroyl and R₂ represents E-p-coumaroyl; and
R₁ and R₂ represent Z-p-coumaroyl; and

b) compounds having the formula V wherein
R₁ and R₂ represents E-p-coumaroyl;
R₁ represents E-p-coumaroyl and R₂ represents Z-p-coumaroyl;
R₁ represents Z-p-coumaroyl and R₂ represents E-p-coumaroyl; and
R₁ and R₂ represent Z-p-coumaroyl.

In another embodiment, the present invention provides a composition comprising each of the following compounds:

a) compounds having the formula IV wherein
R₁ represents E-p-coumaroyl and R₂ represents Z-p-coumaroyl;
R₁ represents Z-p-coumaroyl and R₂ represents E-p-coumaroyl; and
R₁ and R₂ represent Z-p-coumaroyl; and

b) compounds having the formula V wherein
R₁ represents E-p-coumaroyl and R₂ represents Z-p-coumaroyl;
R₁ and R₂ represent Z-p-coumaroyl.

In certain embodiments, the composition comprises a compound of Formula I, IV or V where R₁ and R₂ represent Z-p-coumaroyl. In another embodiment, the composition comprises a mixture of a compounds (for example, a compound of formula IV where R₁ and R₂ represent Z-p-coumaroyl and a compound of formula IV where R₁ and R₂ represent Z-p-coumaroyl).

Compositions of the invention may be used in, but not limited to, pharmaceutical, agricultural or sanitation settings. Depending on the use, the compositions may further contain acceptable carriers. For example, a pharmaceutical composition of the present invention comprises at least one compound of the present invention and may further comprise a pharmaceutically acceptable carrier. Such carriers, buffers, excipients, and antioxidants, flavorings, etc. are known in the art. Compounds of the present invention can be formulated according to known methods for preparing pharmaceutically useful compositions. Formulations are described in detail in a number of sources that are well known and readily available to those skilled in the art. For example, Remington's Pharmaceutical Science by E. W. Martin describes formulations that can be used in connection with the present invention. In general, compositions of the present invention will be formulated such that an effective amount of compound(s) of the present invention are combined with a suitable carrier to facilitate effective administration of the composition.

In accordance with the invention, pharmaceutical compositions comprising, as the active ingredient, an effective amount of one or more of the subject compounds and one or more non-toxic, pharmaceutically acceptable carriers or diluents, can be used by persons of ordinary skill in the art. In addition, the pharmaceutical composition can comprise one or more of the compounds of the present invention as a first active ingredient together with a second or third active ingredient comprising an anti-infective or anti-bacterial compound known in the art.

The most effective mode of administration and dosage regimen will depend upon the severity of the infection and course of that condition, previous therapy, the patient's health status and response, as well as the judgment of the treating physician. Compositions of the invention may be administered to the patient at one time or over a series of treatments.

The present pharmaceutical compositions may comprise a compound of the invention or a derivative or analog or an optical isomer or racemate or tautomer thereof or a pharmaceutically acceptable salt thereof, optionally in admixture with a pharmaceutically acceptable diluent or carrier.

Any of the compounds of the invention, derivatives or analogs can be administered to an animal host, including a human patient, by it self, or in pharmaceutical compositions where it is mixed with suitable carriers or excipient(s) at doses therapeutically effective to treat or ameliorate a bacterial infection. A therapeutically effective dose further refers to that amount of the compound sufficient to result in amelioration of symptoms associated with the infection. Techniques for formulation and administration of the compounds of the instant application may be found in “Remington's Pharmaceutical Sciences,” Mack Publishing Co., Easton, Pa., latest edition.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays. For example, a dose can be formulated in animal models to achieve a circulating concentration range that includes the IC₅₀ (the dose where 50% of the cells show the desired effects) as determined in cell culture. Such information can be used to more accurately determine useful doses in humans.

A therapeutically effective dose refers to that amount of the compound that result in amelioration of symptoms or a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics,” Ch., 1 μl). Dosage amount and interval may be adjusted individually to provide plasma levels of the active moiety which are sufficient to maintain the desired effects.

In cases of local administration or selective uptake, the effective local concentration of the drug may not be related to plasma concentration. The amount of composition administered will, of course, be dependent on the subject being treated, on the subject's weight, the severity of the affliction, the manner of administration and the judgment of the prescribing physician.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations, which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated.

Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion.

Formulations for injection may be presented in unit dosage faun, e.g., in ampoules or in multi dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example) as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions.

Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

Disinfectant/Antiseptic Compositions

Compounds of the present invention can also be used in compositions for use as a topical or surface disinfectant or antiseptic. The antiseptic can be used on a mammal, including a human. The compositions of the present invention may further include herbal or additional disinfectant or antiseptic substances.

Compositions of the present invention may be used in sanitizing a surface, for example in a hospital room or ward. In such cases the composition is applied to the surfaces. The composition may be either a solution or an emulsion in suitable liquid carriers or it may be formulated for spray application. The spray application may also be an electrostatic spray, using an appropriate solvent or solvent system known to those skilled in the art. A spray may also be used, for example, for dispensing a composition of the present invention onto a hand (or other part) of a person. An electrostatic spray application to a hand may be used, with advantage, where a substantially uniform coverage of antiseptic is particularly important e.g. to a surgeon during “scrubbing up” before surgery. The liquids for applying to a surface, by spraying or otherwise, in accordance with the invention may contain, apart from the solvent(s) and/or other liquid carrier(s), other components as necessary or desirable for the intended purpose. Thus, a second or further antiseptic or disinfectant (including from an herbal or other natural origin) may be included, as well as additional substances such as, but not limited to surfactants and fragrances etc. In general, the non-active ingredients of the compositions of the invention may be identical to known compositions for the purpose except that they contain compounds of the present invention in whole or part substitution for one or more of, the other active ingredients.

Compounds or compositions of the present invention may also be included as a component in household detergents, cleansers and creams, for example, washing powders or conditioners and hand gels. Again, the compounds may be included in what are otherwise standard or known compositions for the purpose concerned. The compounds of the present invention may be an extra ingredient or in partial or complete replacement of a standard ingredient. The compositions may already contain a disinfectant or antiseptic and the compounds of the present invention may be added to give an extra or extended antiseptic effect.

In addition, compounds of the present invention may be impregnated into household objects that may be prone to microbial infestation, e.g. dishcloths, plastic soap dishes, surfaces used for the preparation of food. For these purposes, the compounds may be included during manufacture of the object, e.g. in mixtures for plastics moldings or the like, or it may be applied to the object after manufacture, e.g. by soaking dishcloths in compositions of the invention. The presence of the compound(s) at the surface of the object will provide the desired antiseptic effect.

Agricultural

The present invention further provides compositions comprising at least one compound of the present invention for agricultural applications, for example as use in a pesticide (herbicides, insecticides, fungicides, bactericides, molluscicides, and nematicides). For example, compounds and compositions of the present invention are also useful in agricultural applications to protect plants and crops against insects, viruses, fungi, weeds and other undesirable pests.

The compounds and compositions may be applied to a plant, either topically such as by spraying or through watering for systemic applications.

Compounds of this invention will generally be used as a formulation or composition with an agriculturally suitable carrier comprising at least one of a liquid diluent, a solid diluent or a surfactant. The formulation or composition ingredients are selected to be consistent with the physical properties of the active ingredient (composition of the present invention), mode of application and environmental factors such as soil type, moisture and temperature. Useful formulations include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films, and the like which can be water-dispersible (“wettable”) or water-soluble. Active ingredient can be (micro) encapsulated and further formed into a suspension or solid formulation; alternatively the entire formulation of active ingredient can be encapsulated (or “over coated”). Encapsulation can control or delay release of the active ingredient.

Surfactants include, for example, polyethoxylated alcohols, polyethoxylated alkylphenols, polyethoxylated sorbitan fatty acid esters, dialkyl sulfosuccinates, alkyl sulfates, alkylbenzene sulfonates, organosilicones, N,N-dialkyltaurates, lignin sulfonates, naphthalene sulfonate formaldehyde condensates, polycarboxylates, and polyoxyethylene/polyoxypropylene block copolymers. Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, paraffins, alkylbenzenes, alkylnaphthalenes, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, and alcohols such as methanol, cyclohexanol, decanol and tetrahydrofurfuryl alcohol.

Accordingly, the present invention provides a method of controlling pests and/or weeds in a crop by administering compounds or compositions of the present invention to the crop.

### Method of Treating Bacterial Infection

The present invention also provides a method of treating a bacterial infection comprising administering an effective amount of any one of the compounds or a mixture comprising at least one compound of the present invention or compositions of the present invention. In certain embodiments, the bacterial infection is a Staphylococcus aureus infection and in certain embodiments, the bacterial infection is a methicillin or vancomycin resistant Staphylococcus aureus infection.

The present invention also provides methods of treating a bacterial infection in a subject comprising administering an effective amount of at least one compound of the invention. As used herein, treating means either an improvement in condition, reduction of symptoms and even complete eradication of the infection. In certain embodiments, it may be preferable to administer a combination of more than 2 and even up to more than 6 (but not limited to 6) compounds of the present invention. This may help reduce the chances of the bacteria developing resistance to the compounds. The subject may be treated with compounds or compositions of the invention at the same time or may be treated sequentially with different compounds or compositions of the invention.

In certain embodiments, the method of treatment comprises administering a compound of formula I wherein R₁=R₂=E-p-coumaroyl.

In certain embodiments the bacteria infection is caused by Staphylococcus aureus. The Staphylococcus aureus may methicillin-resistant, vancomycin-resistant, or may be any other drug resistant strain.

### α-Glucosidase Inhibitors

Compounds of the invention may also be used as α-glucosidase inhibitors (AGH). This may provide a useful strategy for treating type II diabetes. α-glucosidase inhibitors target enzymes in the small intestine brush border and interfere with the digestion of carbohydrates, achieving better glycemic control. Several natural α-glucosidase inhibitors including acarbose, voglibose, and miglitol are currently used clinically.

The inhibitory activity against α-glucosidase can be measured. Compounds are added to a phosphate buffered solution of α-glucosidase and incubated to allow action of the compound on the α-glucosidase to proceed. A phosphate buffered solution of p-nitrophenyl α-D-glucopyranoside is then added and incubated. The absorbance of each samples is measured at 405 nm with a spectrophotometer. A positive control such as acarbose can be used. Inhibition can be calculated by the following equation:

Inhibition(%)=[1(Aₛₐₘₚₗₑ)/(A_{control})]×100%.

### Methods of Extracting

The present invention also provides a method of extracting the compounds of the invention from a sycamore tree and/or its parts, such as leaves, bark, twigs and small branches. The method comprises mixing sycamore tree parts, such as leaves with a first aqueous rich liphophilic solvent for a length of time sufficient to remove undesired highly hydrophilic compounds. Depending on the mixing conditions and the ratio of leaves to solvent and temperature of the solution, generally about two hours is sufficient. The first aqueous rich liphophilic solvent is removed and a second aqueous rich liphophilic solvent is added and mixed for a time sufficient to extract the compounds. Depending on the mixing conditions and the ratio of leaves to solvent and temperature of the solution, generally about two hours is sufficient. Finally the volume of the aqueous rich liphophilic solvent is reduced to cause precipitation of the compounds of the present invention.

The first aqueous rich liphophilic solvent may be, but is not limited to, EtOH:H₂O, carbon dioxide:H₂O, lower alcohol:H₂O and a mixture of other aliphatics (carbon-hydrogen linear compounds) in H₂O. Aliphatics are known in the art and include, but are not limited to, alkanes, alkenes, and alkynes. In certain embodiments, the first aqueous rich liphophilic solvent comprises EtOH:H₂O (25:75). In certain embodiments, the second aqueous rich liphophilic solvent comprises EtOH:H₂O (75:25).

### EXAMPLES

### Example 1

### Extraction and Isolation

Ten kg of dried leaves, bark, and branches of P. occidentalis of different sizes were extracted at room temperature with ethanol yielding a 400 g extract residue. An aliquot of 50 g of ethanolic extract was loaded onto a 1 kg silica gel column and subjected to vacuum liquid chromatography. Gradient elution was carried out initially with hexane and increasing polarity with hexane:EtOAc, EtOAc, ETOAc:MeOH, MeOH and MeOH:H₂O. A total of nine fractions were obtained including the active fraction EtOAc:MeOH (7 g), from which (4 gm) was further purified on HPLC, C8 column (250×150 mm) by CH₃CN:H₂O:MeOH yielding active fraction (275 mg). The active fraction was further purified using an amino column (250×21.20 mm) using hexane/DCM/MeOH system which purified as 3 well resolved peaks, one of them representing two compounds. Final purification and isolation of the major glycoside compound (1) was carried out by applying isocratic mode on amino column (250×21.20 mm) using DCM:MeOH (25:75) while the final purifications of glycoside (2), (3) and (4) were done using PFP (Pentafluoro phenyl) (250×10 mm) column using ACN:H₂O:MeOH.

The carbohydrate analysis: 0.3 mg of each glycoside alone was hydrolyzed with 2N HCl (1 ml) for 3 hours at 95° C. cooling, neutralize with NH₄OH and then extracted using EtOA_{C}/H₂O. The aqueous layer was dried then dissolved in pyridine (0.3 ml) and 0.1 M in pyridine L-cysteine methyl ester hydrochloride. The reaction mixture was heated for 1 hour at 60° C. then an equal volume of AC₂O was added with continuous heating for extra 1 hour. The acylated thiazolidine derivatives were subjected to GC analysis capillary column: DB-5 ms (30 m×0.25 mm×0.25 μm). The sugars were identified as L-rhamnose as compared with the standard.

### Example 2

### In Vivo MRSA Assay

The in vivo antimicrobial activity for the isolated glycosides is represented in FIGS. 2A and 2B. Mice (CD-1) were purchased from Harlan Inc. and acclimatized up to 5 days before use. Food and water were available ad libitum throughout the study. They were rendered neutropenic by two injections of cyclophosphamide (100 mg/kg) given intraperitoneally, one and four days before infection. A volume of 50 uL suspension of Methicillin-resistant Staphylococcus aureus (1.5×10⁶ live organisms) was injected intramuscularly (1M) into each of the two rear thighs. Each thigh was considered as an individual data point (total of 4 data points for each treatment). These mice were randomly distributed into the control or treatment groups (n=2/group).

Treatment with the test compound (1) was initiated via the intraperitoneal route 2 hrs after thigh inoculation. Control animals were concurrently administered saline (mock treatment) or vancomycin in the same volume as those receiving compound (1). All animals were sacrificed after 48 hours by CO₂ inhalation. Immediately following sacrifice, thigh tissue was collected from the animals, weighed, and homogenized in 5.0 mL of saline. Thigh muscle homogenate was then processed for microbiological assay to determine the number of MRSA cfu per gram of muscle tissue.

FIGS. 2A and 2B provide the in vivo results of compound (1), which demonstrated that in the muscle tissue of untreated control animals (vehicle group) the MRSA cfu/g was 8.02×10⁷. The number of MRSA isolated from vancomycin treated animals was 2.2×10⁷ g of tissue, a reduction of 72% compared to vehicle control. Similarly, the number of MRSA recovered after treatment with compound (1) at 3 mg/kg and 15 mg/kg were 2.6×10⁷ and 1.8×10⁷ respectively, which correspond to a reduction of 67% and 78% as compared to vehicle control. These results confirm the in vitro data and indicate that the test compound has shown equal efficacy compared to vancomycin at the doses tested. To determine the maximum tolerated dose in the preliminary experiment, a maximum of 20 mg/kg of compound (1) was giving IP for two days. No untoward effect was seen in these animals indicating no acute toxicity.

### Example 3

### Cytotoxicity Testing

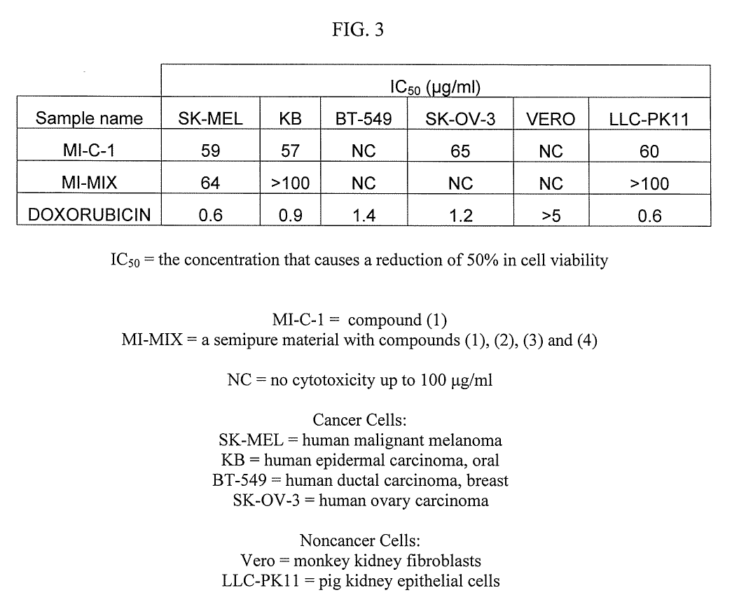

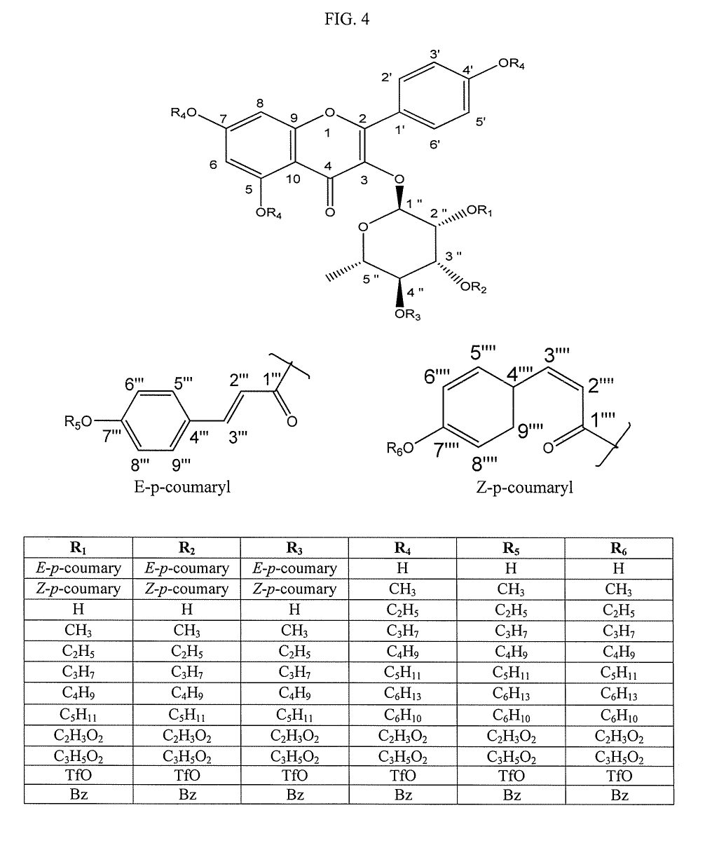

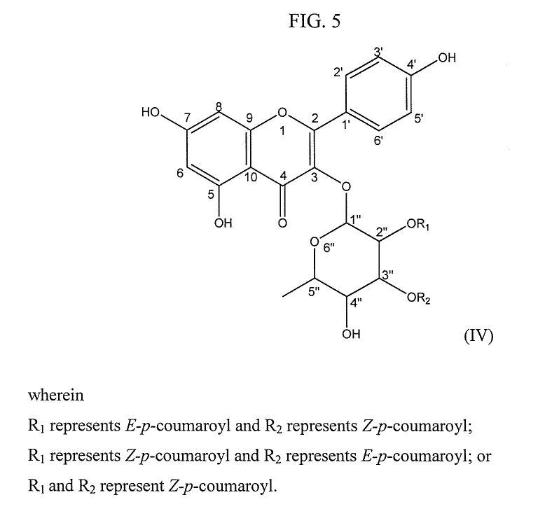

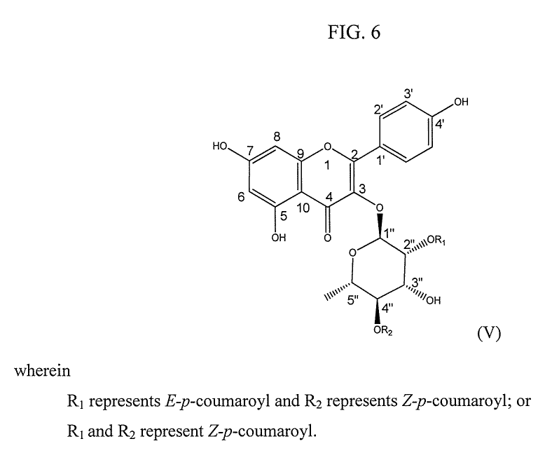

FIG. 3 provides the results of cytotoxicity testing in a panel of cell lines that were incubated with test compounds for 48 hours and cell viability was determined. The cytotoxicity of compound (1) and a mixture of compounds (1), (2), (3) and (4) was tested at high concentrations to determine at what concentration the compounds became cytotoxic. Stock solutions were prepared at 20 mg/ml in DMSO. Cytotoxicity was tested up to a highest concentration of 100 μg/ml. In a routine assay, a stock solution of 2 mg/ml is used and the highest test concentration is 10 μg/ml. The results from the in vitro cytotoxicity testing are set forth in FIG. 3. The results show that the compounds of the present invention are much less cytotoxic (about 100 times less toxic) than doxorubicin, which was used as a positive control for comparing the toxic effect of these compounds.

We claim:

1. A compound of formula (I) 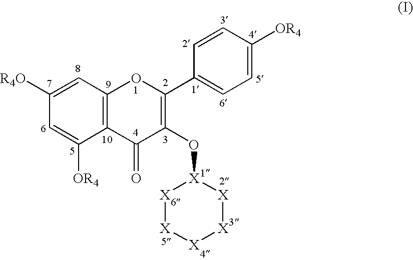
wherein
X_{1″}, X_{2″}, X_{3″}, X_{4″}, X_{5″}, X_{6″} are independently C, COR₁, COR₂, COR₃, CY or N, S or O;
wherein Y is CH₃;
wherein at least one of X_{1″}, X_{2″}, X_{3″}, X_{4″}, X_{5″}, X₆″ is N, S, or O;
wherein R₁, R₂ and R₃ are independently, hydrogen, halogen, hydroxyl, C₆ alkyl, C₁-C₆ alkoxy, C₁-C₆ acyloxy, mono, di or tri-C₁-C₆ alkyl, mono, di or tri-C₁-C₆ N-alkyl, C₁-C₆ alkylthiol, C₁-C₆ acylamino, thiol, nitro, amino, C₁-C₆ alkylsulfonyl, aminosulfonyl, hydroxy sulfonyl (—SO₃H), E-p-coumaroyl, Z-p-coumaroyl;
wherein E-p-coumaroyl has the formula: 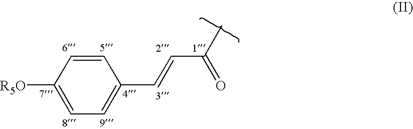
and wherein Z-p-coumaroyl has the formula: 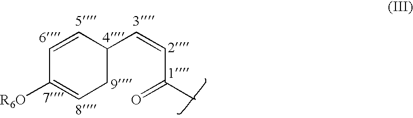
wherein R₄, R₅ and R₆ are independently hydrogen, halogen, hydroxyl, C₆ alkyl, C₁-C₆ alkoxy, C₁-C₆ acyloxy, mono-C₁-C₆ alkyl, di-C₁-C₆ N-alkyl, C₁-C₆ alkylthiol, C₁-C₆ acylamino, thiol, nitro, amino, C₁-C₆ alkylsulfonyl, aminosulfonyl, hydroxy sulfonyl (—SO₃H).

2. The compound of claim 1 wherein
R₁, R₂ and R₃ are independently E-p-coumaroyl, Z-p-coumaroyl, hydrogen, CH₃, C₂H₅, C₃H₇, C₄H₉, C₅H₁₁, C₆H₁₃, C₆H₁₀, C₂H₃O₂, C₃H₅O₂, trifluoromethanesulfonate (CF₃—SO₃—) or benzoyl (C₆H₅—C(═O—); and wherein
R₄, R₅ and R₆ are independently hydrogen, hydrogen, CH₃, C₂H₅, C₃H₇, C₄H₉, C₅H₁₁, C₆H₁₃, C₆H₁₀, C₂H₃O₂, C₃H₅O₂, trifluoromethanesulfonate (CF₃—SO₃—) or benzoyl (C₆H₅—C(═O—).

3. A compound of claim 1 having the formula where 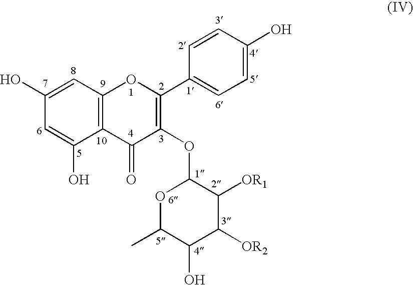
wherein
R₁ represents E-p-coumaroyl and R₂ represents Z-p-coumaroyl;
R₁ represents Z-p-coumaroyl and R₂ represents E-p-coumaroyl; or
R₁ and R₂ represent Z-p-coumaroyl.

4. The compound of claim 3 wherein represents R₁ E-p-coumaroyl and R₂ represents Z-p-coumaroyl.

5. The compound of claim 3 wherein R₁ represents Z-p-coumaroyl and R₂ represents E-p-coumaroyl.

6. The compound of claim 3 wherein R₁ and R₂ represent Z-p-coumaroyl.

7. A method of treating a bacterial infection comprising administering an effective amount of at least one compound of claim 1.

8. A method of treating bacterial infection comprising administering an effective amount of at least two to six compounds of claim 1, wherein administering at least two to six compounds controls or delays the development of bacterial resistance.

9. The method of claim 7 further comprising administering a compound of formula wherein R₁=R₂=E-p-coumaroyl.

10. A method of treating a bacterial infection comprising administering an effective amount of at least one compound of claim 3.

11. The method of claim 7 wherein the bacteria infection is caused by Staphylococcus aureus.

12. The method of claim 11 wherein the Staphylococcus aureus is methicillin resistant, vancomycin resistant or any other drug resistant strain.

13. A pharmaceutical composition comprising at least one compound or mixture of compounds of claim 1 in a pharmaceutically acceptable carrier thereof.

14. A method of extracting the compounds of claim 1 from sycamore tree parts, the method comprising:
a) mixing sycamore tree parts with a first aqueous rich liphophilic solvent for about two hours to remove undesired highly hydrophilic compounds;
b) removing the first aqueous rich liphophilic solvent and adding a second aqueous rich liphophilic solvent and mixing for about two hours to extract the compounds of claim 1; and
c) reducing the volume to precipitate the compounds of the present invention.

15. The method of claim 14 wherein the first aqueous rich liphophilic solvent is selected from the group consisting of EtOH:H₂O, carbon dioxide:H₂O, lower alcohol:H₂Os and aliphatics:H₂O.

16. The method of claim 14 wherein the first aqueous rich liphophilic solvent comprises EtOH:H₂O (25:75).

17. The method of claim 14 wherein the second aqueous rich liphophilic solvent comprises EtOH:H₂O (75:25).

18. A topical or surface disinfectant comprising at least one compound of claim 1 alone or in combination with an additional herbal or disinfectant substance.

19. A topical or surface disinfectant comprising at least one compound of claim 3 alone or in combination with an additional herbal or disinfectant substance.

20. A topical or surface antiseptic comprising at least one compound of claim 1.

21. The antiseptic of claim 20 for use on a mammal.

22. A compound having the formula V where 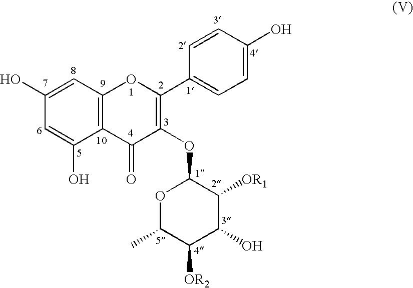
wherein
R₁ represents E-p-coumaroyl and R₂ represents Z-p-coumaroyl; or
R₁ and R₂ represent Z-p-coumaroyl.

23. A composition comprising at least one compound of claim 22 in combination with at least one compound having the formula V wherein
R₁ and R₂ represents E-p-coumaroyl; or
R₁ represents Z-p-coumaroyl and R₂ represents E-p-coumaroyl.

24. A composition comprising at least one compound of claim 1 and at least one compound of claim 22.

25. A composition comprising at least one compound of claim 3 and at least one compound of claim 22.

26. A composition comprising each of the following compounds:
a) compounds having the formula IV wherein
R₁ and R₂ represents E-p-coumaroyl;
R₁ represents E-p-coumaroyl and R₂ represents Z-p-coumaroyl;
R₁ represents Z-p-coumaroyl and R₂ represents E-p-coumaroyl; and
R₁ and R₂ represent Z-p-coumaroyl; and
b) compounds having the formula V wherein
R₁ and R₂ represents E-p-coumaroyl;
R₁ represents E-p-coumaroyl and R₂ represents Z-p-coumaroyl;
R₁ represents Z-p-coumaroyl and R₂ represents E-p-coumaroyl; and
R₁ and R₂ represent Z-p-coumaroyl.

27. A composition comprising each of the following compounds:
a) compounds having the formula IV wherein
R₁ represents E-p-coumaroyl and R₂ represents Z-p-coumaroyl;
R₁ represents Z-p-coumaroyl and R₂ represents E-p-coumaroyl; and
R₁ and R₂ represent Z-p-coumaroyl; and
b) compounds having the formula V wherein
R₁ represents E-p-coumaroyl and R₂ represents Z-p-coumaroyl;
R₁ and R₂ represent Z-p-coumaroyl.

28. A topical or surface disinfectant comprising at least one compound of claim 22 alone or in combination with an additional herbal or disinfectant substance.

29. A topical or surface antiseptic comprising at least one compound of claim 22.

30. The antiseptic of claim 28 for use on a mammal.

31. A topical or surface disinfectant comprising the compounds of claim 26 alone or in combination with an additional herbal or disinfectant substance.
